(19) United States
(12) Patent Application Publication
(10) Pub. No.: US 2003/0158199 A1
(43) Pub. Date: Aug. 21, 2003

(54) Novel compounds for inhibition of Tie-2

(75) Inventors: Frank Stieber, Dortmund (DE); Klaus Hellmuth, Berlin (DE); Herbert Waldmann, Dortmund (DE); Ralph Mazitschek, Karlsruhe (DE); Athanassios Giannis, Karlsruhe (DE)

Correspondence Address: Barbara E. Johnson, WEBB ZIESENHEIM LOGSDON ORKIN & HANSON, P.C., 700 Koppers Building, 436 Seventh Avenue, Pittsburgh, PA, 15219-1818, US

(73) Assignee: KYLIX, B.V., Driebergen (NL)

(21) Appl. No.: 10/057,747

(22) Filed: Jan. 25, 2002

(51) Int. Cl. A61K 31/52; A61K 31/501; A61K 31/4439; A61K 31/427; A61K 31/422; C07D 413/02; C07D 417/02

(52) U.S. Cl. 514/242; 514/252.05; 514/256; 514/340; 514/342; 514/365; 514/374; 544/182; 544/238; 544/333; 546/269.7; 546/271.4; 548/235; 548/202

(57) ABSTRACT

The present invention provides a method of inhibiting or moderating the kinase activity of tyrosine kinases comprising the administration of a compound represented by formula (1) said kinase in sufficient concentration to inhibit or moderate the enzyme activity of said kinase.

### BACKGROUND OF THE INVENTION

Angiogenesis is a multistep process for the formation of new blood vessels from existing vasculature that normally occurs only during embryonic development, breast lactation, endometrail regulation and wound repair. During angiogenesis endothelial cells release enzymes that degrade the basement membrane, migrate through the membrane to form a sprout, and proliferate to extend the vessel (for review see Carmeliet and Jain, 2000).

All of these processes are strictly regulated by factors that either induce or inhibit angiogenesis. When the production and action of these factors is unbalanced, angiogenic factors can be released from tumor cells, migrate to the nearby endothelial cells and induce an angiogenic response cascade. This process is required for the growth of tumors beyond a certain size since they undergo neovascularization and enter a phase of rapid cell growth that may lead to metastasis. Without neovascularizaion tumors enter necrotic and/or apoptotic processes. Increasing vessel density correlates with the likelihood that a patient would develop a metastastic disease (Weidner et al., 1991). This finding illustrates the important role of angiogenesis in cancer.

A number of growth factors are involved in vascular development (reviewed in Yancopoulos et al., 2000). They include at least five members of the vascular endothelium growth factor (VEGF) family, at least four members of the angiopoietin (Ang) family, and at least one member of the large ephrin family. To form functional vessels all of these factors have to act in a coordinated manner. VEGF can initiate vessel formation in adult animals, and Ang-1 further stabilizes and protects the adult vasculature. Their corresponding receptors are exclusively members of the receptor tyrosine kinase (RTK) family of protein kinases.

They are membrane-spanning proteins with an extracellular domain responsible for ligand binding, and a well conserved cytoplasmic tyrosine kinase domain. Signal transduction from the outer to the inner side of the cell is facilitated by a conformational change of the receptor after ligand binding, followed by dimerization and autophosphorylation of the receptor. Autophosphorylation of tyrosines in the activation loop of the tyrosine kinase (TK) domain leads to stimulation of catlytic activity, while autophosphorylation of other tyrosines generates binding sites for proteins with either SH2 or PTB domains. Engagement of these downstream effectors with this autophosphorylation leads to phosphorylation by the receptor which is the starting point for triggering a cascade of downstream signalling events (reviewed in Hubbard, 1999).

The receptors that respond to VEGFs form a family of three closely related RTKs termed VEGFR-1 (Flt-1), VEGFR-2 (KDR or Flk-1) and VEGFR-3 (Flt-4) (reviewed in Tallquist et al., 1999). Their extracellular portion all contain seven immunglobulin-like (Ig) domains and a split intracellular kinase domain. While the major growth and permeability actions of VEGF are mediated by VEGFR-2, growth factor signalling is suppressed by VEGFR-1 because it probably acts as a decoy receptor. Mice lacking VEGFR-2 die between day 8.5 and 9.5 during embryogenesis due to very few enothelial cells and failure to develop a vasculature. Mice lacking VEGFR-1 form excess endothelial cells and disorganized blood vessels also die between E8.5 and E9.5. VEGFR-3 knockout embryos show a cardiovascular failure between E10 and E12 from defects in remodeling the primary vessel networks into larger blood vessels. VEGFR-3 seems to play a role in lymphangiogenesis since its expression is critical for lymphatic vessels (Valtola et al., 1999).

Another group of angiogenic receptors is formed by the two closely related RTKs, Tie-1 (Partanen et al., 1992) and Tie-2 (Ziegler et al., 1993). These are proteins of approximately 125 kD with a single putative transmembrane region. The extracellular domain contains at least three epidermal growth factor (EGF)-like regions of cystein expression, at least two immunglobulin G (IgG)-like domains and at least three regions with fibronectin III-like repeats. The intracellular portion of Tie-2 contains a tyrosine kinase domain pith about 40% sequence identity to that of FGFR-1, PDGFR and c-Kit with the typical motifs for ATP binding (GXGXXG) and tyrosine phosphorylation (HRDLAARN and DFGL).

The Tie receptors are specifically expressed in developing vascular endothelial cells. Embryos deficient in Tie-1 fail to establish structural integrity of vascular endothelial cells, resulting in oedema and subsequently localized haemorrhage. However, analyses of embryos deficient in Tie-2 showed that it is important in angiogenesis, particularly for vascular network formation in endothelial cells, indicating that the structurally related receptor tyrosine kinases Tie-1 and Tie-2 have important but distinct roles in the formation of blood vessels (Dumont et al., 1994; Korhonen et al., 1994; Puri et al., 1995; Sato et al., 1995).

Two ligands for the Tie-2 receptor have been reported. While Angiopoietin-1 (Ang-1) binds and induces the tyrosine phosphorylation of Tie-2, it does not directly promote the growth of cultured endothelial cells but is essential for normal vascular development in the mouse (Davis et al., 1996). Mice engineered to lack Angiopoietin-1 display angiogenic deficits reminiscent of those previously seen in mice lacking Tie-2, demonstrating that Angiopoietin-1 is a primary physiologic ligand for Tie-2 and that it has critical in vivo angiogenic actions that are distinct from VEGF (Suri et al., 1996). Transgenic overexpression of Ang-1 in the skin of mice produces larger, more numerous, and more highly branched vessels (Suri et al,, 1998). This finding supports a more direct role of Ang-1 in angiogenesis and vascular remodelling.

Angiopoietin-2 (Ang-2) was identified by homology screening and showed to be a naturally occurring antagonist for Ang-1 and Tie-2. Therefore, transgenic overexpression of Ang-2 disrupts blood vessel formation in the mouse embryo. In adult mice and humans, Ang-2 is expressed only at sites of vascular remodeling (Maisonpierre et al., 1991).

Interestingly, mice embryos knocked out for VEGFR-2 (Flk-1) show lethal defects in vasculogenesis that are earlier than a corresponding disruption of Tie-2 . This and the other findings described above indicate that the VEGF/VEGFR signalling system seems to be necessary for the early stages of vascular development, while the Ang-1/Tie-2 system is required for the later stages of vascular remodeling.

These results raise the possibility that angiopoietins can be used, alone or in combination with VEGF, to promote therapeutic angiogenesis. On the other hand, blocking or moderating of the Tie receptor system may block or moderate angiogenesis and further proliferation of tumor cells. By in situ hybridization only a weak Tie-1 mRNA signal was obtained from adult skin, except during wound healing, when the proliferating capillaries in the granulation tissue contained abundant Tie RNA (Korhonen et al., 1992). However, capillaries and medium-sized vessels within cutaneous and brain metastases of melanoma were strongly positive for Tie mRNA. A Tie-specific amplified cDNA band was obtained by RT-PCR from melanoma metastases but not from normal skin. These results suggest a role for the Tie receptor system in angiogenesis associated with melanoma metastases (Kaipainen et al., 1994).

Administration of Ad-ExTek, a soluble adenoviral expressed extracellular domain of Tie-2, inhibited tumor metastasis when delivered at the time of surgical excision of primary tumors in a clinically relevant mouse model of tumor metastasis (Lin et al., 1998). The inhibition of Tie-2 function by ExTek may be a consequence of sequestration of the angiopoietin ligand and/or heterodimerisation with the native Tie-2 receptor. This study demonstrates that disruption of Tie-2 signalling pathways, first, may be well tolerated in healthy organisms and, second, may provide therapeutic benefit.

### SUMMARY OF THE INVENTION

A compound of formula I

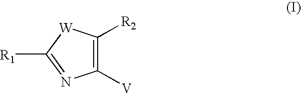

wherein V is H or

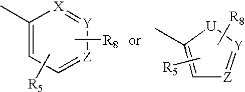

R₁ can be independently H, alkyl, alkenyl, cycloalkyl, heteroalkyl, aryl, heteroaryl, arylalkyl, alkylaryl, N—R₆R₇, N—(CO)R₆R₇, N—R₆(CO)R₇ or N—(CO)—O—R₆R₇,

R₈ can be independently H, alkyl, alkenyl, cycloalkyl, heteroalkyl, aryl, heteroaryl, aryl, heteroaryl, alkyl, alkylaryl, N—R₃R₄, N—(CO)R₃R₄, N—R₃(CO)R₄, N—(CO)—O—R₃R₄, O—R₃, CO—R₃, CO—OR₃ or O—CO—R₃,

R₂, R₆, can be independently H, alkyl, alkenyl, cycloalkyl, heteroalkyl, aryl, heteroaryl, arylalkyl, alkylaryl, Br, Cl, F, CF₃,

R₃, R₄, R₆, R₇ can be independently H, alkyl, alkenyl, cycloalkyl, heteroalkyl, aryl, heteroaryl, arylalkyl, alkylaryl, COOR₅ and CO—R₅, and may for a ring structure,

X, Y, Z can be independently CH or N, and

U can be independently S or NH,

W can be independently NH, O or S, and

racemic-diastereomeric mixtures, optical isomers, and pharmaceutically acceptable salts thereof.

The present invention provides a method of inhibiting or moderating the kinase activity of tyrosine kinases comprising the administration of a compound represented by formula (1) said kinase in sufficient concentration to inhibit or moderate the enzyme activity of said kinase.

The present invention further provides the use of compounds in pharmaceutical compositions with a pharmaceutically acceptable carrier or excipient. These pharmaceutical compositions can be administered to individuals to slow or halt the process of angiogenesis in angiogenesis-aided diseases or cancer in general.

Definitions of the Various Terms

Listed below definitions of various terms used to describe the compounds of the instant invention. These definitions apply to the terms as they are used throughout the specification (useless they are otherwise limited in specific instances) either individually or as part of a larger group. It should be noted that any heteroatom with unsatisfied valances is assumed to have the hydrogen-atom to satisfy the valances.

The term “alkyl” or “alk” refers to a monovalent alkane (hydrocarbon) derived radical containing from 1 to 20 carbon atoms unless otherwise defined. An alkyl group is an optionally substituted straight, branched or cyclic saturated hydrocarbon group. When substituted, alkyl groups may substituted with R at any available point of attachment. R is defined as R₁. When the alkyl group is said to be substituted with alkyl group this is used interchangeably with “branched alkyl group”. Exemplary unsubstitute such groups may include but are not limited to methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include but are not limited to one or more of the following groups: halogen (such as F, Cl, Br, I), haloalkyl (such as CCl₃ and CF₃), alkoxy, alkylthio, hydroxy, carboxy (—COOH), alkyloxycarbonyl (—C(O)R), alkylcarbonyloxy (—OCOR), amino (—NH₂), alkylamino, dialkylamino, carbamoyl (—NHCOOR— or —OCONHR—), urea (—NHCONHR) or thiol (—SH). R is defined as R₆.

Alkyl groups as defined may also comprise one or more carbon to carbon double bonds or one or more carbon to carbon triple bonds.

The term “alkenyl” refers to a hydrocarbon radical straight, branched or cyclic containing from 2 to 20 carbon atoms and at least one carbon to carbon double bond.

The term “alkynyl” refers to a hydrocarbon radical straight, branched or cyclic containing from 2 to 20 carbon atoms and at least one carbon to carbon triple bond.

Cycloalkyl is a specie of alkyl containing from 3 to 15 carbon atoms, without alterning or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents may include but are not limited to one or more of the following groups: halogen, alkyl, alkoxy, alkyl hydroxy, amino, alkylamino, dialkylamino, nitro, cyano, thiol and/or alkylthio.

Cycloalkenyl is a specie of alkenyl containing from 3 to 15 carbon atoms, without alterning or resonating double bonds between carbon atoms and at least one carbon to carbon double bond. It may contain from 1 to 4 rings. Exemplary unsubstituted such groups include cyclopropyl, cyclobutyl, cyclopentyl, cyclohexyl, adamantyl, etc. Exemplary substituents may include but are not limited to one or more of the following groups: halogen, alkyl, alkoxy, alkyl hydroxy, amino, alkylamino, dialkylamino, nitro, cyano, thiol and/or alkylthio.

Cycloalkynyl is a specie of alkyl containing from 3 to 15 carbon atoms, without alterning or resonating double bonds between carbon atoms and at least one carbon to carbon triple bond. It may contain from 1 to 4 rings. Exemplary unsubstituted such groups include cyclopropyl, cyclobutyl, cyclopentyl, cyclohexyl, adamantyl, etc. Exemplary substituents may include but are not limited to one or more of the following groups: halogen, alkyl, alkoxy, alkyl hydroxy, amino, alkylamino, dialkylamino, nitro, cyano, thiol and/or alkylthio.

The term “heterocycloalkyl” refers to a cycloalkyl group (nonaromatic) in which one to three of the carbon atoms in the ring are replaced by a heteroatom selected from O, S or N.

The term “heterocycloalkenyl” refers to a cycloalkenyl group (nonaromatic) in which one to three of the carbon atoms in the ring are replaced by a heteroatom selected from O, S or N.

The term “heterocycloalkynyl” refers to a cycloalkynyl group (nonaromatic) in which one to three of the carbon atoms in the ring are replaced by a heteroatom selected from O, S or N.

The term “aryl” refers to monocyclic, bicyclic, tricyclic or tetracyclic aromatic rings, e.g. phenyl, substituted phenyl and the like, as well as groups which are fused, e.g. naphtyl, substituted naphtyl, phenanthrenyl or substituted phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Aryl groups may optionally be substituted with one or more groups including, but not limited to halogen, alkyl, alkoxy, hydroxy, carboxy, carbamoyl, alkyloxycarbonyl, nitro, trifluoromethyl, amino, NH—R₇, NR₆R₇, cycloalkyl, cyano, alkyl S(O)ₘ (m=0,1,2), SO₂—NR₆R₇, NR₆—SO₂—R₇, or thiol.

The term “heteroaryl” refers to a monocyclic aromatic hydrocarbon group having 5 or 6 ring atoms, a bicyclic aromatic group having 8 to 10 atoms, or a tricyclic aromatic group having 11 to 14 atoms containing at least one heteroatom, O, S, or N, in which a carbon or nitrogen atom is the point of attachment, and in which one to three additional carbon atoms is optionally replaced by a heteroatom selected from O, N, or S, said heteroaryl group being optionally substituted as described herein. Exemplary heteroaryl groups may include but are not limited to the following: thienyl, furyl, pyrrolyl, pyridinyl, imidazolyl, oxazolyl, pyrrolidinyl, piperidinyl, thiazolyl, pyrazinyl, pyridazinyl, pyrimidinal, triazinylazepinyl, indolyl, isoindolyl, quinolinyl, isoquinolinyl, benzothiazolyl, benzoxazolyl, benzimidazolyl, benzoxadiazolyl, benzofuranzanyl tetrahydropyranyl and the like. Exemplary substituents may include but are not limited to one or more of the following: halogen, alkyl, alkoxy, hydroxy, carboxy, carbamoyl, alkyloxycarbonyl, trifluoromethyl, cycloalkyl, nitro, cyano, amino, NH—R₇, NR₆R₇, alkyl-S(O)ₘ (m=0,1,2), or thiol and the like.

The term “arylalkyl”, as used herein, denotes an aromatic ring bonded to an alkyl group as described above.

The term “alkylaryl”, as used herein, denotes an alkyl group bonded to an aromatic ring as described above.

The terms “alkoxy” or “alkylthio”, as used herein, denote an alkyl group as described above bonded through an oxygen linkage (—O—) or a sulfur linkage (—S—), respectively.

The terms “alkenyloxyl” or “alkenylthio”, as used herein, denote an alkenyl group as described above bonded through an oxygen linkage (—O—) or a sulfur linkage (—S—), respectively.

The terms “alkynyloxy” or “alkynylthio”, as used herein, denote an alkynyl group as described above bonded through an oxygen linkage (—O—) or a sulfur linkage (—S—), respectively.

The terms “cycloalkoxy” or “cycloalkylthio”, as used herein, denote an cycloalkyl group as described above bonded through an oxygen linkage (—O—) or a sulfur linkage (—S—), respectively.

The terms “cycloalkenyloxy” or “cycloalkenylthio”, as used herein, denote a cycloalkenyl group as described above bonded through an oxygen linkage (—O—) or a sulfur linkage (—S—), respectively.

The terms “cycloalkynyloxy” or “cycloalkynylthio”, as used herein, denote a cycloalkynyl group as described above bonded through an oxygen linkage (—O—) or a sulfur linkage (—S—), respectively.

The terms “heterocycloalkoxy” or “heterocycloalkylthio”, as used herein, denote a heterocycloalkyl group as described above bonded through an oxygen linkage (—O—) or a sulfur linkage (—S—) respectively.

The terms “heterocycloalkenyloxy” or “heterocycloalkenylthio”, as used herein, denote a heterocycloalkenyl group as described above bonded through an oxygen linkage (—O—) or a sulfur linkage (—S—), respectively.

The terms “heterocycloalkynyloxy” or “heterocycloalkynylthio”, as used herein, denote an heterocycloalkynyl group as described above bonded through an oxygen linkage (—O—) or a sulfur linkage (—S—), respectively.

The terms “aryloxy” or “aryllthio”, as used herein, denote an aryl group as described above bonded through an oxygen linkage (—O—) or a sulfur linkage (—S—), respectively.

The terms “heteroalkyloxy” or “heteroalkyllthio”, as used herein, denote an heteroalkyl group as described above bonded through an oxygen linkage (—O—) or a sulfur linkage (—S—), respectively.

The terms “heteroalkenyloxy” or “heteroalkenyllthio”, as used herein, denote an heteroalkenyl group as described above bonded through an oxygen linkage (—O—) or a sulfur linkage (—S—), respectively.

The terms “heteroalkynyloxy” or “heteroalkynyllthio”, as used herein, denote an heteroalkynyl group as described above bonded through an oxygen linkage (—O—) or a sulfur linkage (—S—), respectively.

The terms “arylalkoxy” or “arylalkylthio”, as used herein, denote an arylalkyl group as described above bonded through an oxygen linkage (—O—) or a sulfur linkage (—S—), respectively.

The terms “alkylaryloxy” or “alkylaryllthio”, as used herein, denote an alkylaryl group as described above bonded through an oxygen linkage (—O—) or a sulfur linkage (—S—), respectively.

The terms “alkylamin” or “alkyloxycarbonyl”, as used herein, denote an alkyl group as described above bonded through an nitrogen linkage (—N—) or denotes an alkoxy group bonded through a carbonyl group, respectively.

The terms “alkenylamin” or “alkenyloxycarbonyl”, as used herein, denote an alkenyl group as described above bonded through an nitrogen linkage (—N—) or denotes an alkoxy group bonded through a carbonyl group, respectively.

The terms “alkynylamin” or “alkynyloxycarbonyl”, as used herein, denote an alkynyl group as described above bonded through an nitrogen linkage (—N—) or denotes an alkoxy group bonded through a carbonyl group, respectively.

The terms “cycloalkamin” or “cycloalkyloxycarbonyl”, as used herein, denote an cycloalkyl group as described above bonded through an nitrogen linkage (—N—) or denotes an alkoxy group bonded through a carbonyl group, respectively.

The terms “cycloalkenylamin” or “cycloalkenyloxycarbonyl”, as used herein, denote a cycloalkenyl group as described above bonded through an nitrogen linkage (—N—) or denotes an alkoxy group bonded through a carbonyl group, respectively.

The terms “cycloalkynylamin” or “cycloalkynyloxycarbonyl”, as used herein, denote a cycloalkynyl group as described above bonded through an nitrogen linkage (—N—) or denotes an alkoxy group bonded through a carbonyl group, respectively.

The terms “heterocycloalkamin” or “heterocycloalkyloxycarbonyl”, as used herein, denote a heterocycloalkyl group as described above bonded through an nitrogen linkage (—N—) or denotes an alkoxy group bonded through a carbonyl group, respectively.

The terms “heterocycloalkenylamin” or “heterocycloalkenyloxycarbonyl”, as used herein, denote a heterocycloalkenyl group as described above bonded through an nitrogen linkage (—N—) or denotes an alkoxy group bonded through a carbonyl group, respectively.

The terms “heterocycloalkynylamin” or “heterocycloalkynyloxycarbonyl”, as used herein, denote an heterocycloalkynyl group as described above bonded through an nitrogen linkage (—N—) or denotes an alkoxy group bonded through a carbonyl group, respectively.

The terms “arylamin” or “arylloxycarbonyl”, as used herein, denote an aryl group as described above bonded through an nitrogen linkage (—N—) or denotes an alkoxy group bonded through a carbonyl group, respectively.

The terms “heteroalkylamin” or “heteroalkylloxycarbonyl”, as used herein, denote an heteroalkyl group as described above bonded through an nitrogen linkage (—N—) or denotes an alkoxy group bonded through a carbonyl group, respectively.

The terms “heteroalkenylamin” or “heteroalkenylloxycarbonyl”, as used herein, denote an heteroalkenyl group as described above bonded through an nitrogen linkage (—N—) or denotes an alkoxy group bonded through a carbonyl group, respectively.

The terms “heteroalkynylamin” or “heteroalkynylloxycarbonyl”, as used herein, denote an heteroalkynyl group as described above bonded through an nitrogen linkage (—N—) or denotes an alkoxy group bonded through a carbonyl group, respectively.

The terms “arylalkamin” or “arylalkyloxycarbonyl”, as used herein, denote an arylalkyl group as described above bonded through an nitrogen linkage (—N—) or denotes an alkoxy group bonded through a carbonyl group, respectively.

The terms “alkylarylamin” or “alkylarylloxycarbonyl”, as used herein, denote an alkylaryl group as described above bonded through an nitrogen linkage (—N—) or denotes an alkoxy group bonded through a carbonyl group, respectively.

The term “heteroatom” means O, S or N, selected on an independent basis.

The term “halogen” refers to chlorine, bromine, fluorine or iodine.

When a functional group is termed “protected”, this means that the group is in modified form to preclude undesired side reactions at the protected site. Suitable protecting groups of the compounds of the present invention will be recognized from the present application taking into account the level of skill in the art, and with reference to standard textbooks, such as Greene, T. W. et al., Protective Groups in Organic Synthesis, Wiley, N.Y. (1991).

Suitable examples of salts of the compounds according to the invention with inorganic or organic acids are hydrochloride, hydrobromide, hydrosulfate, sulfate, hydrophosphate, phosphate and the like. Salts which are unsuitable for pharmaceutical uses but which can be employed, for example, for the isolation or purification of free compounds (1) or (2) or their acceptable salts, are also included.

Suitable salts of carboxylic groups, if present, like sodium, potassium, lithium or magnesium or other pharmaceutically acceptable salts are also included.

All stereoisomers of the compounds of the instant invention are contemplated, either in a mixture or in pure or substantially pure form. The definition of the compounds according to the invention embraces all possible stereoisomers and their mixtures. It very particularly embraces the racemic forms and the isolated optical isomers having the specified activity. The racemic forms can be dissolved by physical methods, such as, for example fractional crystallisation, separation or crystallisation of diastereomeric derivatives or separation by chiral column chromatography. The individual optical isomers can be obtained from the racemates by conventional methods, such as, salt formation with an optically active acid followed by crystallization.

It should be understood that solvates (e.g, hydrates) of the compounds of formula (1) and (2) are also within the scope of the present invention. Methods of solvation are generally known in the art. Accordingly, the compounds of the instant invention may be in the free or hydrate form, and may be optained by methods exemplified.

### DETAILED DESCRIPTION OF THE INVENTION

The present invention relates to a new class of compounds that block or moderate kinase activity of tyrosine kinases.

An embodiment of the invention relates to a new class of compounds that block or moderate kinase activity of the tyrosine kinase Tie-2. These compounds are defined in claim 1.

Follwing IC50 values were determined in the RTK ELISA using recombinant kinase domains of receptor tyrosine kinases which were expressed in baculovirus infected insect cells.

| | | IC50 [μM] | | |
|---|---|---|---|---|
| Number | Structure | Tie-2 | KDR | c-Met |
| 2 | 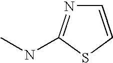 | >50 | 5 | >50 |
| 3 | 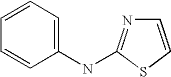 | 10 | 3.5 | >50 |
| 4 | 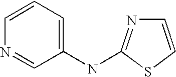 | >50 | 5 | >25 |
| 5 | 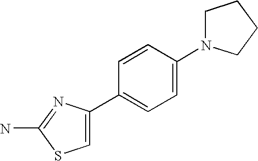 | 2.5 | 5 | 5 |
| 6 | 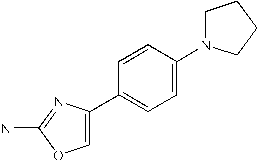 | 5 | >25 | >50 |
| 7 | 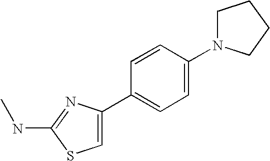 | 3 | 7 | >50 |
| 8 | 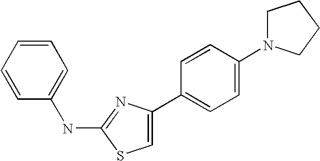 | 5 | 2.5 | >25 |
| 9 | 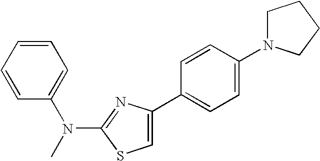 | 2.5 | >25 | >25 |
| 10 | 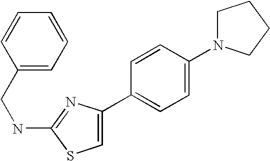 | 5 | >50 | >50 |
| 11 | 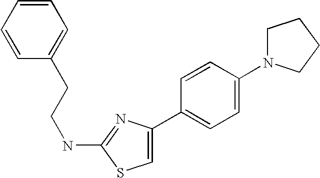 | >50 | | |
| 12 | 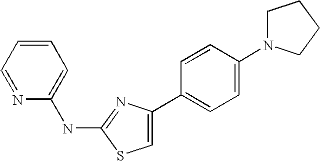 | >50 | | |
| 13 | 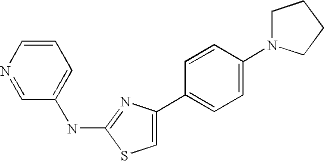 | 1 | 2 | >10 |
| 14 | 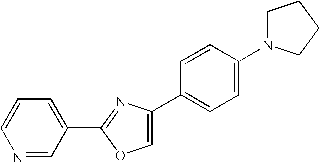 | 5 | >50 | >25 |
| 15 | 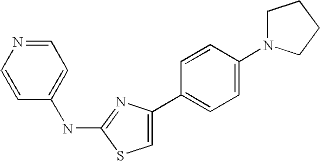 | 1 | 5 | >10 |
| 16 | 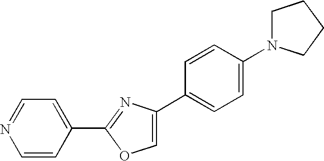 | >50 | >50 | >50 |
| 17 | 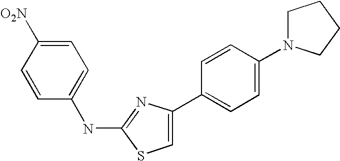 | 2 | >10 | >25 |
| 18 | 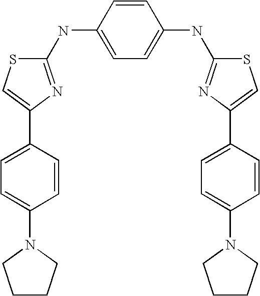 | >10 | >10 | >50 |
| 19 | 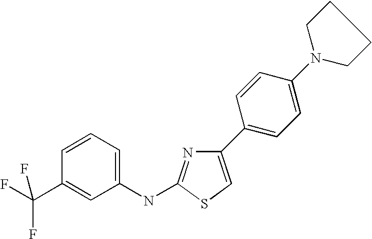 | 2 | 2 | >50 |
| 20 | 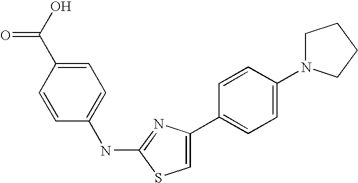 | <1 | 1 | >10 |
| 21 | 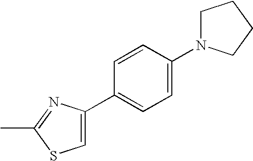 | 2.5 | 10 | >50 |
| 22 | 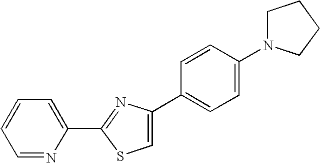 | <1 | 1 | 3 |
| 23 | 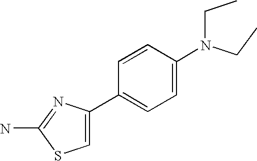 | 5 | 2 | >50 |
| 24 | 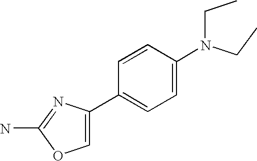 | 5 | 1.5 | >50 |
| 25 | 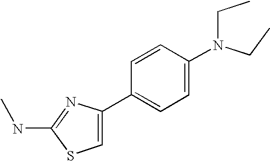 | 1 | 5 | >50 |
| 26 | 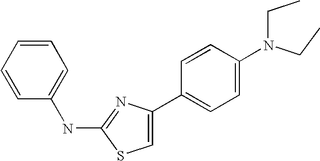 | 2 | 2 | >25 |
| 27 | 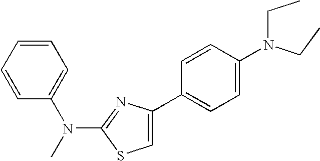 | >50 | >50 | >50 |
| 28 | 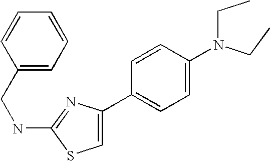 | >50 | >50 | >50 |
| 29 | 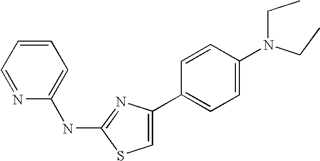 | >50 | >50 | >50 |
| 30 | 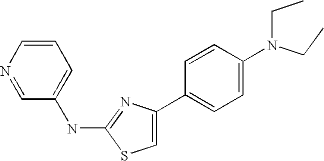 | 0.8 | 3 | >50 |
| 31 | 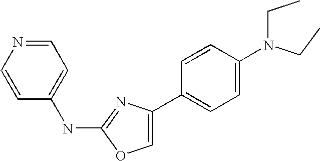 | >50 | >25 | >50 |
| 32 | 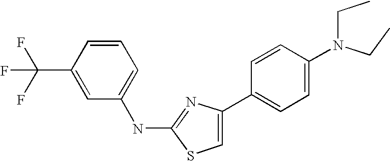 | 3 | 5 | >50 |
| 33 | 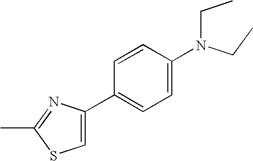 | 5 | 5 | >50 |
| 34 | 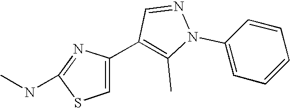 | 1.5 | 1.5 | >50 |
| 35 | 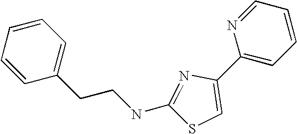 | 1 | 1 | 2 |
| 36 | 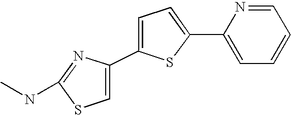 | >20 | >10 | >50 |
| 37 | 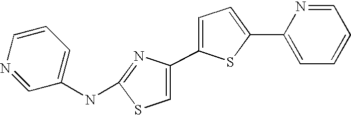 | 2.5 | >50 | >50 |

### Synthesis

### Synthesis of methyl-thiazol-2-yl-amine Hydrochloride (2)

9.1 mg (0.1 mmol) N-methylthiourea and 12.9 μl (0.1 mmol) chloracetaldehyde solution in water (approx. 55%) were dissolved in 10 ml ethanol and stirred for 15 h at 60° C. The solvent was removed and the residue was dried in vacuo.

Yield: 17.4 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=0.55 min, purity >95%.

HPLC-MS: 115.1 (M+H).

### Synthesis of phenyl-thiazol-2-yl-amine Hydrochloride (3)

15.2 mg (0.1 mmol) phenylthiourea and 12.9 μl (0.1 mmol) chloracetaldehyde solution in water (approx. 55%) were dissolved in 10 ml ethanol and stirred for 15 h at 60° C. The solvent was removed and the residue was dried in vacuo.

Yield: 20.3 mg

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.22 min, purity >90%.

GCMS: 176 (M⁺).

### Synthesis of pyridin-3-yl-thiazol-2-yl-amine Hydrochloride (4)

15.3 mg (0.1 mmol) 3-pyridylthiourea and 12.9 μl (0.1 mmol) chloracetaldehyde solution in water (approx. 55%) were dissolved in 10 ml ethanol and stirred for 15 h at 60° C. The solvent was removed and the residue was dried in vacuo.

Yield: 20.4 mg

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=0.85 min, purity >80%.

HPLC-MS: 178 (M+H).

### Synthesis of 4-(4-pyrrolidin-1-yl-phenyl)-thiazol-2-ylamine Hydrobromide (5)

134 mg (0.5 mmol) 2-bromo-4′-(1-pyrrolidino)-acetaphenone and 38 mg (0.5 mmol) thiourea were dissolved in 5 ml ethanol and stirred for 15 h at 60° C. The solvent was removed and the residue was dried in vacuo.

Yield: 139.2 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.83 min, purity >95%

HPLC-MS: 246 (M+H).

### Synthesis of 4-(4-pyrrolidin-1-yl-phenyl)-oxazol-2-ylamine Hydrobromide (6)

134 mg (0.5 mmol) 2-bromo-4′-(1-pyrrolidino)-acetophenone and 30 mg (0.5 mmol) urea were dissolved in 5 ml ethanol and stirred for 15 h at 60° C. The solvent was removed and the residue was dried in vacuo.

Yield: 163.7 mg

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.71 min, purity >85%.

### Synthesis of methyl-[4-(4-pyrrolidin-1-yl-phenyl)-thiazol-2-yl]-amine Hydrobromide (7)

805 mg (3 mmol) 2-bromo-4′-(1-pyrrolidino)-acetophenone and 270 mg (3 mmol) N-methylthiourea were dissolved in 20 ml ethanol and stirred for 15 h at 60° C. The solvent was removed and the residue was dried in vacuo.

Yield: 1.17 g.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.94 min, purity >95%.

GCMS: 259 (M⁺).

### Synthesis of phenyl-[4-(4-pyrrolidin-1-yl-phenyl)-thiazol-2-yl]-amine Hydrobromide (8)

805 mg (3 mmol) 2-bromo-4′-(1-pyrrolidino)-acetophenone and 570 mg (3 mmol) phenylthiourea were dissolved in 20 ml ethanol and stirred for 15 h at 60° C. The solvent was removed and the residue was dried in vacuo.

Yield: 1.31 g.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.63 min, purity >90%.

HPLC-MS: 322 (M+H).

### Synthesis of methyl-phenyl-[4-(4-pyrrolidin-1-yl-phenyl)-thiazol-2-yl]-amine Hydrobromide (9)

268 mg (1 mmol) 2-bromo-4′-(1-pyrrolidino)-acetophenone and 166 mg (1 mmol) N-methyl-N-phenylthiourea were dissolved in 20 ml ethanol and stirred for 15 h at 60° C. The solvent was removed and the residue was dried in vacuo.

Yield: 442 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.94 min, purity >95%.

HPLC-MS: 336 (M+H).

### Synthesis of benzyl-[4-(4-pyrrolidin-1-yl-phenyl)-thiazol-2-yl]-amine Hydrobromide (10)

268 mg (1 mmol) 2-bromo-4′-(1-pyrrolidino)-acetophenone and 166 mg (1 mmol) benzylthiourea were dissolved in 20 ml ethanol and stirred for 15 h at 60° C. The solvent was removed and the residue was dried in vacuo.

Yield: 409 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.40 min, purity >98%.

### Synthesis of phenylethyl-[4-(4-pyrrolidin-1-yl-phenyl)-thiazol-2-yl]-amine Hydrobromide (11)

536 mg (2 mmol) 2-bromo-4′-(1-pyrrolidino)-acetophenone and 360 mg (2 mmol) 2-phenylethylthiourea were dissolved in 5 ml ethanol and stirred for 15 h at 60° C. The solvent was removed and the residue was dried in vacuo.

Yield: 850 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ₌2.51 min, purity >99%

GCMS: 349 (M⁺).

### Synthesis of phenylethyl-[4-(4-pyrrolidin-1-yl-phenyl)-thiazol-2-yl]-amine Hydrobromide (12)

268 mg (1 mmol) 2-bromo-4′-(1-pyrrolidino)-acetophenone and 153 mg (1 mmol) 2-pyridylthiourea were dissolved in 5 ml ethanol and stirred for 15 h at 60° C. The solvent was removed and the residue was dried in vacuo.

Yield: 382 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.18 min, purity >95%

GCMS: 322 (M⁺).

### Synthesis of phenylethyl-[4-(4-pyrrolidin-1-yl-phenyl)-thiazol-2-yl]-amine Hydrobromide (13)

268 mg (1 mmol) 2-bromo-4′-(1-pyrrolidino)-acetophenone and 153 mg (1 mmol) 3-pyridylthiourea were dissolved in 20 ml ethanol and stirred for 15 h at 60° C. The solvent was removed and the residue was dried in vacuo.

Yield: 397 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.40 min, purity >98%.

GCMS: 322 (M⁺).

### Synthesis of 3-[4-(4-pyrrolidin-1-yl-phenyl)-oxazol-2-yl]-pyridine Hydrobromide (14)

134 mg (0.5 mmol) 2-bromo-4′-(1-pyrrolidino)-acetophenone and 61 mg (0.5 mmol) nicotinamide were dissolved in 10 ml ethanol and stirred for 15 h at 90° C. The solvent was removed and the residue was dried in vacuo.

Yield: 183.5 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.66 min, purity >90%.

¹H-NMR (CDCl₃, 500 MHz): d=9.88 (s, 1H, arom. CH), 9.03 (d, ³J(H,H)=7 Hz, 2H, arom. CH), 8.98 (d, ³J(H,H)=7 Hz, 2H, arom. CH), 7.92 (m, 1H, arom. CH), 7.73 (d, ³J(H,H)=9 Hz, 2H, arom. CH), 6.39 (d, ³J(H,H)=9 Hz, 2H, arom. CH), 6.33 (s, 1H, oxazole-CH), 3.23 (m, 4H, N—CH₂—C), 1.89 (m, 4H, N—CH₂—CH₂—C).

### Synthesis of pyridin-4-yl-[4-(4-pyrrolidin-1-yl-phenyl)-thiazol-2-yl]-amine Hydrobromide (15)

134 mg (0.5 mmol) 2-bromo-4′-(1-pyrrolidine)-acetophenone and 76.6 mg (0.5 mmol) 4-pyridylthiourea were dissolved in 15 ml ethanol and stirred for 15 h at 60° C. The solvent was removed and the residue was dried in vacuo.

Yield: 201.1 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.77 min, purity >80%

### Synthesis of 4-[4-(4-pyrzolidin-1-yl-phenyl)-oxazol-2-yl]-pyridine Hydrobromide (16)

134 mg (0.5 mmol) 2-bromo-4′-(1-pyrrolidino)-acetophenone and 61 mg (0.5 mmol) isonicotinamide were dissolved in 10 ml ethanol and stirred for 15 h at 90° C. The solvent was removed and the residue was dried in vacuo.

Yield: 195.3 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.65 min, purity >98%.

¹H-NMR (CDCl₃, 500 MHz): d=9.14 (d, ³J(H,H)=7 Hz, 2H, arom. CH), 8.50 (d, ³J(H,H)=7 Hz, 2H, arom. CH), 1.87 (d, ³J(H,H)=9 Hz, 2H, arom. CH), 6.68 (d, ³J(H,H)=9 Hz, 2H, arom. CH), 6.40 (s, 1H, oxazole-CH), 3.37 (q, ³J(H,H)=7 Hz, 4H, N—CH₂—C), 1.99 (m, 4H, N—CH₂—CH₂—C).

### Synthesis of N,N′-bis-[4-(4-pyrrolidin-1-yl-phenyl)-thiazol-2-yl]-benzene-1,4-diamine Dihydrobromide (17)

268 mg (1 mmol) 2-bromo-4′-(1-pyrrolidino)-acetophenone and 197 mg (1 mmol) 4-nitrophenylthiourea were dissolved in 20 ml ethanol and stirred for 15 h at 60° C. The solvent was removed and the residue was dried in vacuo.

Yield: 270.3 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.79 min, purity >99%.

HPLC-MS: 367 (M+H).

### Synthesis of N,N′-bis-[4-(4-pyrrolidin-1-yl-phenyl)-thiazol-2-yl]-benzene-1,4-diamine Dihydrobromide (18)

268 mg (1 mmol) 2-bromo-4′-(1-pyrrolidino)-acetaphenone and 113 g (0.5 mmol) 1,4-phenylenbisthiourea were dissolved in 20 ml ethanol and stirred for 15 h at 60° C. The solvent was removed and the residue was dried in vacuo.

Yield: 376.4 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.80 min, purity >95%.

HPLC-MS: 565 (M+H).

### Synthesis of [4-(4-pyrrolidin-1-yl-phenyl)-thiazol-2-yl]-(3-trifluoromethyl-phenyl)-amine Hydrobromide (19)

27.5 mg (0.1 mmol) 2-bromo-4′-(1-pyrrolidino)-acetophenone and 22.0 mg (0.1 mmol) 3-(trifluormethyl)-phenylthiourea were dissolved in 10 ml ethanol and stirred for 15 h at 60° C. The solvent was removed and the residue was dried in vacuo.

Yield: 51 mg.

HPLC (Column: Xterra, MS C18, 5 μm, 4.6*100 mm; 3 ml/min; 10% to 90% B gradient in 3.5 min., hold 0.5 min at 90% B, Solvent A: water+0.1% trifluoroacetic acid, Solvent B: 100% acetonitrile+0.1% trifluoroacetic acid, UV: 214 nm, 254 nm, 301 nm): rₜ=3.51 min, purity >80%.

HPLC-MS: 390 (M+H).

### Synthesis of 4-[4-(4-pyrrolidin-1-yl-phenyl)-thiazol-2-ylamino]-benzoic Acid Hydrobromide (20)

27.5 mg (0.1 mmol) 2-bromo-4′-(1-pyrrolidino)-acetophenone and 19.6 mg (0.1 mmol) 4-carboxyphenylthiourea were dissolved in 10 ml Ethanol and stirred for 15 h at 60° C. The solvent was removed and the residue was dried in vacuo.

Yield: 46.2 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.56 min, purity >98%.

HPLC-MS: 366 (M+H).

### Synthesis of 2-methyl-4-(4-pyrrolidin-1-yl-phenyl)-thiazole Hydrobromide (21)

134 mg (0.5 mmol) 2-bromo-4′-(1-pyrrolidino)-acetophenone and 38 mg (0.5 mmol) thioacetamide were dissolved in 10 ml ethanol and stirred for 15 h at 90° C. The solvent was removed and the residue was dried in vacuo.

Yield: 170.6 mg.

HPLC (Column: Xterra, MS C18, 5 μl, 4.6*100 mm; 3 ml/min; 10% to 90% B gradient in 3.5 min Solvent A: water+0.1% trifluoroacetic acid, Solvent B: 100% acetonitrile+0.1% trifluoroacetic acid, UV: 214 nm, 254 nm, 301 nm): rₜ=2.36 min, purity >90%.

HPLC-MS: 245 (M+H).

### Synthesis of 3-[4-(4-pyrrolidin-1-yl-phenyl)-thiazol-2-yl]-pyridine Hydrobromide (22)

134 mg (0.5 mmol) 2-bromo-4′-(1-pyrrolidino)-acetophenone and 69 mg (0.5 mmol) thionicotinamide were dissolved in 10 ml ethanol and stirred for 15 h at 90° C. The solvent was removed and the residue was dried in vacuo.

Yield: 204.7 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.02 min, purity >90%.

GC-MS: 307 (M⁺).

### Synthesis of 4-(4-diethylamino-phenyl)-thiazol-2-ylamine Hydrobromide (23)

136 mg (0.5 mmol) 2-bromo-4′-(diethylamino)-acetophenone and 38 mg (0.5 mmol) thiourea were dissolved in 10 ml ethanol and stirred for 15 h at 60° C. The solvent was removed and the residue was dried in vacuo.

Yield: 168.9 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=0.94 min, purity >95%.

GC-MS: 247 (M⁺).

### Synthesis of 4-(4-diethylamino-phenyl)-oxazol-2-ylamine Hydrobromide (24)

136 mg (0.5 mmol) 2-bromo-4′-(diethylamino)-acetophenone and 30 mg (0.5 mmol) urea were dissolved in 5 ml ethanol and stirred for 15 h at 60° C. The solvent was removed and the residue was dried in vacuo.

Yield: 159.4 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.68 min, purity >90%.

HPLC-MS: 270 (M+K).

### Synthesis of [4-(4-diethylamino-phenyl)-thiazol-2-yl]-methyl-amine Hydrobromide (25)

54.1 mg (0.2 mmol) 2-bromo-4′-(diethylamino)-acetophenone and 18 mg (0.2 mmol) N-methylthiourea were dissolved in 10 ml ethanol and stirred for 15 h at 60° C. The solvent was removed and the residue was dried in vacuo.

Yield: 64.3 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.12 min. purity >90%.

GC-MS: 261 (M⁺).

### Synthesis of [4-(4-Diethylamino-phenyl)-thiazol-2-yl]-phenyl-amine Hydrobromide (26)

54.1 mg (0.2 mmol) 2-bromo-4′-(diethylamino)-acetophenone and 38 mg (0.2 mmol) phenylthiourea were dissolved in 10 ml ethanol and stirred for 15 h at 60° C. The solvent was removed and the residue was dried in vacuo.

Yield: 78.3 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.08 min, purity >90%.

GC-MS: 323 (M⁺).

### Synthesis of [4-(4-diethylamino-phenyl)-thiazol-2-yl]-methyl-phenyl-amine Hydrobromide (27)

27.0 mg (0.1 mmol) 2-bromo-4′-(diethylamino)-acetophenone and 16.6 mg (0.1 mmol) N-methyl-N-phenylthiourea were dissolved in 10 ml ethanol and stirred for 15 h at 60° C. The solvent was removed and the residue was dried in vacuo.

Yield: 42 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.24 min, purity >99%.

GC-MS: 337 (M⁺).

### Synthesis of benzyl-[4-(4-diethylamino-phenyl)-thiazol-2-yl]-amine Hydrobromide (28)

27.0 mg (0.1 mmol) 2-bromo-4′-(diethylamino)-acetophenone and 16.6 mg (0.1 mmol) benzylthiourea were dissolved in 10 ml ethanol and stirred for 15 h at 60° C. The solvent was removed and the residue was dried in vacuo.

Yield: 44 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.86 min, purity >95%

GC-MS: 337 (M⁺).

### Synthesis of [4-(4-diethylamino-phenyl)-thiazol-2-yl)-pyridin-2-yl-amine Hydrobromide (29)

270 mg (1 mmol) 2-bromo-4′-(diethylamino)-acetophenone and 153 mg (1 mmol) 2-pyridylthiourea were dissolved in 10 ml ethanol and stirred for 15 h at 60° C. The solvent was removed and the residue was dried in vacuo.

Yield: 443 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.49 min, purity >95%.

HPLC-MS: 325 (M+H).

### Synthesis of [4-(4-diethylamino-phenyl)-thiazol-2-yl]-pyridin-2-yl-amine Trifluoracetic Acid (30)

108 mg (0.4 mmol) 2-bromo-4′-(diethylamino)-acetophenone and 56 mg (0.4 mmol) 3-pyridylthiourea were dissolved in 20 ml ethanol and stirred for 15 h at 60° C. The solvent was removed and the residue was dried in vacuo. The crude product was purified by preparative HPLC.

Yield: 56 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.30 min. purity >98%.

GC-MS: 324 (M⁺).

### Synthesis of diethyl-[4-(2-pyridin-4-yl-oxazol-4-yl)-phenyl]-amine Hydrobromide (31)

136 mg (0.5 mmol) 2-bromo-4′-(diethylamino)acetophenone and 61 mg (0.5 mmol) isonicotinamide were dissolved in 20 ml ethanol and stirred for 15 h at 90° C. The solvent was removed and the residue was dried in vacuo.

Yield: 160.2 mg.

HPLC (Column: Xterra, MS C18, 5 μm, 4.6*100 mm; 3 ml/min; 10,% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.67 m, purity >95%.

¹H-NMR (CDCl₃, 500 MHz): d =9.10 (d, ³J(H,H)=7 Hz, 2H, arom. CH), 8.49 (d, ³J(H,H)=7 Hz, 2H, arom. CH), 7.84 (d, ³J(H,H)=9 Hz, 2H, arom. CH), 6.81 (d, ³J(H,H)=9 Hz, 2H, arom. CH), 6.35 (s, 1H, oxazole-CH), 3.47 (q, ³J(H,H)=7 Hz, 4H, N—CH₂—C), 1.14 (t, 6H, N—CH₂—CH₃.

### Synthesis of [4-(4-diethylamino-phenyl)-thiazol-2-yl -(3-trifluoromethyl-phenyl)-amine Hydrobromide (32)

27.1 mg (0.1 mmol) 2-bromo-4′-(diethylamino)-acetophenone and 22.0 mg (0.1 mmol) 3-(trifluormethyl)-phenylthiourea were dissolved in 10 ml ethanol and stirred for 15 h at 60° C. The solvent was removed and the residue was dried in vacuo.

Yield: 50.9 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=2.59 min, purity >99%.

GC-MS: 391 (M⁺).

### Synthesis of diethyl-[4-(2-methyl-thiazol-4-yl)-phenyl]-amine Hydrobromide (33)

136 mg (0.5 mmol) 2-bromo-4′-(diethylamino)-acetophenone and 38 mg (0.5 mmol) thioacetamide were dissolved in 10 ml ethanol and stirred for 15 h at 90° C. The solvent was removed and the residue was dried in vacuo.

Yield: 164.5 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.62 min, purity >95%.

HPLC-MS: 247 (M+H).

GC-MS: 246 (M⁺).

### Synthesis of methyl-[4-(5-methyl-1-phenyl-1H-pyrazol-4-yl)-thiazol-2-yl]-amine Hydrochloride (34)

27.9 mg (0.1 mmol) 2-bromo-1-[3-(4-chlorophenyl)-5-isoxazolyl]-1-ethanone and 9.0 mg (0.1 mmol) N-methylthiourea were dissolved in 10 ml ethanol and stirred for 15 h at 60° C. The solvent was removed and the residue was dried in vacuo.

Yield: 24.7 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.70 min, purity >99%.

GC-MS: 270 (M⁺).

### Synthesis of phenylethyl-(4-pyridin-2-yl-thiazol-2-yl)-amine Hydrobromide (35)

28.0 mg (0.1 mmol) 2-bromo-1-(2-pyridinyl)-1-ethanone and 18.0 mg (0.1 mmol) 2-phenylethylthiourea were dissolved in 10 ml ethanol and stirred for 15 h at 60° C. The solvent was removed and the residue was dried in vacuo.

Yield: 45.7 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.95 min, purity >90%.

GC-MS: 232 (M+H).

### Synthesis of methyl-[4-(5-pyridin-2-yl-thiophen-2-yl)-thiazol-2-yl]-amine Hydrobromide (36)

26.6 mg (0.1 mmol) 2-bromo-1-(5-(2-pyidiny)-2-thienyl]-1-ethanone and 9.0 mg (0.1 mmol) N-methylthiourea were dissolved in 10 ml ethanol and stirred for 15 h at 60° C. The solvent was removed and the residue was dried in vacuo.

Yield: 35.6 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.59 min, purity >95%.

GC-MS: 273 (M⁺).

### Synthesis of pyridin-3-yl-[(4-(5-pyridin-2-yl-thiophen-2-yl)-thiazol-2-yl]-amine Hydrobromide (37)

26.6 mg (0.1 mmol) 2-bromo-1-[5-(2-pyridinyl)-2-thienyl]-1-ethanone and 15.3 mg (0.1 mmol) 3-pyridylthiourea were dissolved in 10 ml ethanol and stirred for 15 h at 60° C. The solvent was removed and the residue was dried in vacuo.

Yield: 39.9 mg.

HPLC (Column: Xterra, MS C18, 5 μm, 4.6*100 mm; 3 ml/min; 10% to 90% B gradient in 3.5 min. Solvent A: water+0.1% trifluoroacetic acid, Solvent B: 100% acetonitrile+0.1% trifluoroacetic acid, UV: 214 nm, 254 nm, 301 nm): rₜ=1.59 min, purity >98%.

HPLC-MS: 337 (M+H).

### 1. EXAMPLES

### Example 1

Receptor Tyrosine Kinase Assay

The following in vitro assays are used to determine the ability of different compounds to inhibit the transfer of phosphate groups onto tyrosine residues of downstream substrates. The level of phosphorylation is measured by a monoclonal antibody which is specific for phosphorylated tyrosine residues in an enzyme-linked immunosorbent assay (ELISA).

Materials:

The following recombinant kinases were expressed in baculovirus infected insect cells Sf9 and purchased from ProQuinase, Freiburg:

1.) GST-Tie2/Tek (aa 771-1124)

2.) GST-KDR (aa807-1356)

3.) GST-cMet (aa956-1390)

4.) GST-FGFR1 (aa400-822)

5.) GST-IGF1R (aa905-1337)

6.) GST-cKit (aa544-976)

7.) GST-cAbl (aa118-535)

8.) GST-His6-ErbB2 (aa679-1255)

9.) GST-FLT4 (aa725-1298)

The following recombinant kinase was expressed in baculovirus infected insect cells and purchased from MoBiTec, Göttingen:

10.) Src, partially purified (Panvera P2903)

The following reagents and supplies were used:

96-well LIA places (Greiner 655074)

Poly-Glu-Tyr 4:1 (Sigma P0275)

Adenosin Triphosphate (Sigma A2383)

Dimethylsulfoxide DMSO (Roth A994.2)

Mouse monoclonal antiphosphotyrosin antibody PY20 coupled to horseradish peroxidase (Calbiochem 525320)

Bovine Serum Albumine (BSA) (Calbiochem 12659)

PBS buffer:

137 mM Sodium chloride (Roth 3957.1)

3 mM Potassium chloride (Roth 6781.1)

1.5 mM Potassium dihydrogenphosphate (Roth 3904.1)

8.2 mM Disodium hydrogenphosphate (Roth P030.2)

Sodium ortho vanadate (Sigma S6508)

Manganese dichloride tetrahydrate (Roth T881.1)

HEPES (Roth 9105.2)

Tween 20 (Roth A9127.1)

BM Chemoluminescent ELISA Substrate (Roche 1582950)

Procedure:

If not otherwise indicated all steps are performed at room temperature.

1.) Coat wells of ELISA plate with 10 mg/ml Poly-Glu-Tyr 4:1 in 100 μl/well PBS buffer (137 mM NaCl, 3 mM KCl, 1.5 mM KH₂PO₄, 8.2 mM Na₂HPO₄) overnight at 4° C.

2.) Wash 2 times for 5 min each with PBS+0.05% Tween20

3.) Kinase assay:

a) Add 5-30 ng/well kinase in 50 μl/well kinase buffer (100 mM HEPES pH 7.4, 100 mM NaCl, 0.1 mM Na₃VO₄)

b) Add 25 μl/well compound (50 and 5 μM) in 5% DMSO

c) Add 25 μl/well 100 μM ATP in 40 mM MnCl₂

d) Incubate 30 min

4.) Wash 3 times for 5 min each with PBS+0.05% Tween20

5.) Add anti phosphotyrosin antibody/HRP 1:10.000 in 100 μl/well PBS+0.05% Tween+0.1 BSA, incubate for 1 h

6.) Wash 3 times for 5 min each with PBS+0.05% Tween20

7.) Chemoluminescence reaction:

a.) Premix 25 μl/well BM CLS Solution 1 with 0.25 μl/well BM CLS Solution 2

b.) Preincubate for 15 min

c.) Add 25 μl/well PBS

d.) Add 50 μl/well substrate solution to microtiter wells

e.) Incubate for at least 1 min

8.) Detect chemoluminescence signals in Tecan Genios reader

f.) Mode: luminescence

g.) Integration time: 100 ms

h.) Enhancement factor: 125-150

i.) Shaking time: 5 s

### Example 2

Generation of Ligand

Materials:

293T cells

Dulbeccos modified eagle medium (DMEM) (Life Technologies)

Fetal Calf Serum (Life Technologies)

Cell culture tissue dishes (Greiner)

Escort Transfection Reagent (Sigma)

Procedure:

293T cells are plated at 1×106 cells per well in a six well plate in DMEM medium supplemented with 10% fetal calf serum (FCS), incubated overnight at 37° C. and transfected with 1 μg plasmid DNA of pCB ANG1 by the Escort transfection reagent according to the manufacturer's protocol. After one day medium is changed to DMEM without FCS, and cell culture supernatants are harvested by centrifugation after additional 3 days.

### Example 3

Cell-Based Assay

Materials:

Human umbilical vein endothelial cells (HUVEC) (Promocell C-12200)

Endothelial cell growth (ECG) medium (Promocell C-22010) supplemented with:

2% Petal Calf Serum

0.4% Endothelial Cell Growth Fator (ECGF)

0.1 ng/ml Epidermal Growth factor (EGF)

1 μg/ml Hydrocortison

1 ng/ml bascic Fibroblast Growth Factor (bFGF)

50 ng/ml Amphotericin B

50 μg/ml Gentamicin

N199 medium (Life Technologies)

Detach Kit (Promocell C-41210) contains:

HepesBSS

Trypsin/EDTA solution

Trypsin neutralisation solution (TNS)

Cell culture tissue dishes (Greiner)

PBS buffer:

RIPA buffer:

20 mM Tris/HCl pH 7,5

150 M NaCl

2 mM EDTA

1% Triton X100

1% SDS

0,5% deoxycholat (DOC)

10 gylycerol

4× SDS sample buffer:

250 mM disodium hydrogenphosphate/sodium dihydrogenphosphate pH 7.0

8% SDS

40% glycerol

20% mercaptoethanol

0.01% bromophenol blue

PVDF membranes Immobilon-P (Millipore)

Bovine Serum Albumine (BSA) (Calbiochem 12659)

Tween 20 (Roth A9127.1)

Mouse monoclonal antiphosphotyrosin antibody PY20 coupled to horseradish peroxidase (Calbiochem 525320)

Rabbit polyclonal anti-Tie-2 antibody C-20 (Santa Cruz Biotechnology sc-324)

Goat anti rabbit IgG secondary antibody coupled to horseradish peroxidase (Dianova 111-035-003)

Enhanced chemoluminescence detection kit Supersignal Pico (Pierce 37070)

Procedure:

Endothelial cells e.g. HUVECs between passage 2 and 12 are plated at between 2×105 and 1×106 cells per well in a six well plate in supplemented ECG medium. After 24 to 48 hours the medium is changed to M199 medium containing increasing concentrations of the inhibitory compound in the individual wells. The cells are incubated at 37° C. and then treated with cell culture supernatants containing the ligand for 5 to 30 min.

Afterwards the cells are placed on ice, washed once with 1 ml PBS, lysed by the addition of 300 μl RIPA buffer and removed from the plate by a cell scraper. The suspension is transferred into a microcentrifuge tube, sonicated for 5 sec and boiled after addition of 100 μl 4× SDS sample buffer for 5 min at 95° C.

30 μl of the lysate are run on a 8% SDS-PAGE gel. The separated proteins are then transferred to PVDF membranes according to the manufacturer's instructions for Western blotting.

The blots are blocked with PBS/0.05% Tween 20/1% BSA for 1 h at room temperature, incubated with either antiphosphotyrosin or anti-Tie-2 antibody diluted 1:2000 in PBS/Tween for 1 hour and washed 3 times with PBS/Tween. In the second case the blot is incubated with a goat anti rabbit IgG secondary antibody/HRP conjugate diluted 1:4000 in PBS/Tween. After washing 3 times in PBS/Tween the blot is developed by the ECL method according to the manufacturer's instruction.

Literatur

Carmeliet, P., and Jain, R. K. (2000). Angiogenesis in cancer and other diseases. Nature 407, 249-257.

Davis, S., Aldrich, T. H., Jones, P. F., Acheson, A., Compton, D. L., Jain, V., Ryan, T. E., Bruno, J., Radziejewski, C., Maisonpierre, P. C., and Yancopoulos, G. D. (1996). Isolation of angiopoietin-1, a ligand for the TIE2 receptor, by secretion-trap expression cloning. Cell 87, 1161-1169.

Dumont, D. J., Gradwohl, G., Fong, G. H., Puri, M. C., Gertsenstein, M., Auerbach, A., and Breitman, M. L. (1994). Dominant-negative and targeted null mutations in the endothelial receptor tyrosine kinase, tek, reveal a critical role in vasculogenesis of the embryo. Genes and Development 8, 1897-1909.

Hubbard, S. R. (1999). Structural analysis of receptor tyrosine kinases. Prog Biophys Mol Biol 71, 343-358.

Kaipainen, A., Vlaykova, T., Hatva, E., Bohling, T., Jekunen, A., Pyrhonen, S., and Alitalo, K. (1994). Enhanced expression of the tie receptor tyrosine kinase mesenger RNA in the vascular endothelium of metastatic melanomas Cancer Res 54, 6571-6577.

Korhonen, J., Partanen, J., Armstrong, E., Vaahtokari, A., Elenius, K., Jalkanen, M., and Alitalo, K. (1992). Enhanced expression of the tie receptor tyrosine kinase in endothelial cells during neovascularization. Blood 80, 2548-2555.

Korhonen, J., Polvi, A., Partanen, J., and Alitalo, K. (1994). The mouse tie receptor tyrosine kinase gene, expression during embryonic angiogenesis. Oncogene 9, 395-403.

Lin, P., Buxton, J. A., Acheson, A., Radziejewski, C., Maisonpierre, P. C., Yancopoulos, G. D., Channon, K. M., Hale, L. P., Dewhirst, M., W., George, S. E., and Peters, K. G. (1998). Antiangiogenic gene therapy targeting the endothelium-specific receptor tyrosine kinase Tie2. Proc Natl Acad Sci USA 95, 8829-8834.

Maisonpierre, P. C., Suri, C., Jones, P. F., Bartunkova, S., Wiegand, S. J., Radziejewski, C., Compton, D., McClain, J., Aldrich, T. H., Papadopoulos, N., et al. (1997). Angiopoietin-2, a natural antagonist for Tie2 that disrupts in vivo angiogenesis. Science 277, 55-60.

Partanen, J., Armstrong, E., Makela, T. P., Korhonen, J., Sandberg, M., Renkonen, R., Knuutila, S., Huebner, K., and Alitalo, K. (1992). A novel endothelial cell surface receptor tyrosine kinase with extracellular epidermal growth factor homology domains. Mol Cell Biol 12, 1698-1707.

Puri, M. C., Rossant, J., Alitalo, K., Bernstein, A., and Partanen, J. (1995). The receptor tyrosine kinase TIE is required for integrity and survival of vascular endothelial cells. Embo J 14, 5884-5891.

Sato, T. N., Tozawa, Y., Deutsch, U., Wolburg-Buchholz, K., Fujiwara, Y., Gendron-Maguire, M., Gridley, T., Wolburg, H., Risau, W., and Qin, Y. (1995). Distinct roles of the receptor tyrosine kinases Tie-1 and Tie-2 in blood vessel formation. Nature 376, 70-74.

Suri, C., Jones, P. F., Patan, S., Bartunkova, S., Maisonpierre, P. C., Davis, S., Sato, T. N., and Yancopoulos, G. D. (1996). Requisite role of angiopoietin-1, a ligand for the TIE2 receptor, during embryonic angiogenesis. Cell 87, 1171-1180.

Suri, C., McClain, J., Thurston, G., McDonald, D. M., Zhou, H., Oldmixon, E. H., Sato, T. N., and Yancopoulos, G. D. (1998). Increased vascularization in mice overexpressing angiopoietin-1. Science 282, 468-471.

Tallquist, M. D., Soriano, P., and Klinghoffer, R. A. (1999). Growth factor signaling pathways in vascular development. Oncogene 18, 7917-7932.

Valtola, R., Salven, P., Heikkila, P., Taipale, J., Joensuu, H., Rehn, M., Pihlajaniemi, T., Weich, H., deWaal, R., and Alitalo, K. (1999). VEGFR-3 and its ligand VEGF-C are associated with angiogenesis in breast cancer. Am J Pathol 154, 1381-1390.

Weidner, N., Semple, J. P., Welch, W. R., and Folkman, J. (1991). Tumor angiogenesis and metastasis-correlation in invasive breast carcinoma. N Engl J Med 324, 1-8.

Yancopoulos, G. D., Davis, S., Gale, N. W., Rudge, J. S., Wiegand, S. J., and Holash, J. (2000). Vascular-specific growth factors and blood vessel formation. Nature 407, 242-248.

Ziegler, S. F., Bird, T. A., Schneringer, J. A., Schooley, K. A., and Baum, P. R. (1993). Molecular cloning and characterization of a novel receptor protein tyrosine kinase from human placenta. Oncogene 8, 663-670.

1. A compound of formula I 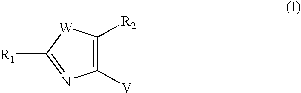
wherein V is H or 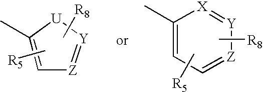
R₁ can be independently H, alkyl, alkenyl, cycloalkyl, heteroalkyl, aryl, heteroaryl, arylalkyl, alkylaryl, N—R₆R₇, N—(CO)R₆R₇, N—R₆(CO)R₇ or N—(CO)—O—R₆R₇,
R₈ can be independently H, alkyl, alkenyl, cycloalkyl, heteroalkyl, aryl, heteroaryl, arylalkyl, alkylaryl, N—R₃R₄, N—(CO)R₃R₄, N—R₃(CO)R₄, N—(CO)—O—R₃R₄, O—R₃, CO—R₃, CO—OR₃ or O—CO—R₃,
R₂, R₅, can be independently H, alkyl, alkenyl, cycloalkyl, heteroalkyl, aryl, heteroaryl, arylalkyl, alkylaryl, Br, Cl, F, CF₃,
R₃, R₄, R₆, R₇ can be independently H, alkyl, alkenyl, cycloalkyl, heteroalkyl, aryl, heteroaryl, arylalkyl, alkylaryl, COOR₅ and CO—R₅, and may for a ring structure,
X, Y, Z can be independently CH or N, and
U can be independently S or NH,
W can be independently NH, O or S, and
racemic-diastereomeric mixtures, optical isomers, and pharmaceutically acceptable salts thereof.

2. Compound as claimed in claim 1, wherein W is O or S, preferably S.

3. Compound as claimed in claim 1 or 2, wherein R₁ is N—R₆R₇.

4. Compound as claimed in claim 1-3, wherein R₂ is H.

5. Compound as claimed in claim 1-4, wherein X, Y, Z is CH.

6. Coumpound as claimed in claim 5, wherin R₃ and R₄ are alkyl or form a N-hetero aryl or alkyl ring.

7. Compound as claimed in claim 1-6, wherein
U is S and Y and Z are C or
U is NR₃, Y is N and Z are C.

8. Compound 1-37.

9. A method of inhibiting one or more protein kinase activity by using a compound of claim 1-8 in vitro or in cell culture.

10. A pharmaceutical composition comprising a pharmaceutically acceptable diluent or carrier and a compound of claim 1-8.

11. Pharmaceutical composition comprising an additive and a compound of claim 1-8.

12. Use of a compound of claim 1-8-as medicament.

13. Use of compound of claim 1-8-as an inhibitor of protein kinase activity.

14. Use of compound as claimed in claim 13 wherein said kinase is selected from the group of tyrosine kinases consisting of Tie-2, KDR, c-Met, FGFR-1, IGF-1R, c-Kit, Flt-4, ErbB-2, c-Ab1, c-Src, and oncogenic variants thereof.

15. Use of a compound as claimed in claim 13 wherein said kinase is a serine/threonine kinase.

16. Use of a compound of claim 1-8 to inhibit the progression of a disease state in a patient.

17. Use of a compound as claimed in claim 13 wherein the disease is selected from the group of cancer, venous malformations and angiogenesis dependent disorders.
